(19) United States
(12) Patent Application Publication
(10) Pub. No.: US 2017/0285018 A1
(43) Pub. Date: Oct. 5, 2017

(54) SENSOR, METHOD OF FORMING A SENSOR AND USE THEREOF

(71) Applicant: Cambridge Display Technology, Ltd., Godmanchester (GB)

(72) Inventors: RICHARD WILSON, Cambridge (GB); Jonathan Behrendt, Bedford (GB)

(73) Assignee: Cambridge Display Technology, Ltd., Godmanchester (GB)

(21) Appl. No.: 15/475,313

(22) Filed: Mar. 31, 2017

(30) Foreign Application Priority Data
Mar. 31, 2016 (GB) GB1605489.2

(51) Int. Cl. G01N 33/543; B05D 3/00; G01N 27/22; B05D 1/00

(52) U.S. Cl. CPC G01N 33/5438; B05D 1/005; B05D 3/007; G01N 27/226

(57) ABSTRACT

A method of forming a sensor comprising a single layer or multilayer structure; a fluorinated layer having a fluorinated surface on the single layer or multiple layer structure; and a receptor having a fluorinated group on the fluorinated surface, the method comprising treating the fluorinated surface with a surfactant and either depositing the receptor having a fluorinated group onto the fluorinated surface from a formulation comprising one or more solvents in which the receptor is dissolved or dispersed, or depositing a fluorinated compound comprising a fluorinated group onto the fluorinated surface from a formulation comprising one or more solvents in which the fluorinated compound is dissolved or dispersed, and reacting the fluorinated compound or a derivative thereof with a receptor comprising a reactive group to form the receptor having the fluorinated group.

### RELATED APPLICATIONS

This application claims the benefits under 35 U.S.C. §119(a)-(d) or 35 U.S.C. §365(b) of British application number GB1605489.2, filed Mar. 31, 2016, the entirety of which is incorporated herein by reference.

### FIELD OF THE INVENTION

The invention relates to sensors, in particular biosensors, use of said sensors and methods of forming said sensors.

### BACKGROUND OF THE INVENTION

A wide range of sensors for detection of an analyte are known.

US 2013/0071289 discloses a sensor comprising a transistor having a coupling/stabilization layer covering at least part of the semiconductor layer of the transistor.

Katz et al, “Label-free brain injury biomarker detection based on highly sensitive large area organic thin film transistor with hybrid coupling layer”, Chem. Sci., 2014, 5, 416 discloses an OTFT biosensor for sensing of glial fibrillary acidic protein (GFAP) having a CYTOP passivation layer, a vapour-deposited layer of tetratetracontane (C₄₄H₉₀) to fill residual pinholes in CYTOP layer and a layer of PS-block-PAA polymer having anti-GFAP immobilized thereon.

Cai et al, “Immobilization of antimicrobial peptide IG-25 onto fluoropolymers via fluorous interactions and click chemistry”, ACS Appl Mater Interfaces. 2013 Dec. 26; 5(24): 12789-12793

Pohl et al, “Fluorous-Based Carbohydrate Microarrays”, J. Am. Chem. Soc. 2005, 127, 13162-13163 discloses certain fluorous tagged sugars deposited as spots on a Teflon/epoxy mixture coated on a microscope slide. Binding to fluorescein isothiocyanate-labeled jack bean lectin concanavalin A (FITC-ConA) was observed using a fluorescent slide scanner.

It is an object of the invention to provide a method of immobilising a receptor on a surface.

It is a further object of the invention to provide a sensor having a simple structure.

It is a further object of the invention to provide a sensor providing an electrical response indicating presence of a target analyte.

### SUMMARY OF THE INVENTION

In a first aspect, the invention provides a method of forming a sensor comprising a single layer or multilayer structure; a fluorinated layer having a fluorinated surface on the single layer or multiple layer structure; and a receptor having a fluorinated group on the fluorinated surface, the method comprising treating the fluorinated surface with a surfactant and either:

depositing the receptor having a fluorinated group onto the fluorinated surface from a formulation comprising one or more solvents in which the receptor is dissolved or dispersed, or
depositing a fluorinated compound comprising a fluorinated group onto the fluorinated surface from a formulation comprising one or more solvents in which the fluorinated compound is dissolved or dispersed, and reacting the fluorinated compound or a derivative thereof with a receptor comprising a reactive group to form the receptor having the fluorinated group.

In a second aspect, the invention provides a sensor comprising:

a structure configured to detect a change in an electrical property in a layer thereof;
a fluorinated layer on a surface of the structure; and
a receptor bound to a fluorinated group on an outer surface of the fluorinated layer.

In a third aspect the invention provides a sensor comprising:

a structure configured to detect a change in an optical property in a layer thereof;
a fluorinated layer on a surface of the structure; and
a receptor bound to a fluorinated group on an outer surface of the fluorinated layer.

In a fourth aspect the invention provides a method of forming a sensor according to the second or third aspect, the method comprising the step of contacting the outer surface of the fluorinated layer with either a formulation comprising the receptor having the fluorinated group bound thereto, or a formulation comprising a fluorinated compound, and reacting the fluorinated compound or a derivative thereof with a receptor comprising a reactive group to form the receptor bound to the fluorinated group.

In a fifth aspect the invention provides a method for detecting a target analyte, the method comprising the step of contacting a sensor according to the second or third aspect with an analyte fluid, preferably an analyte liquid, and measuring a response from the sensor.

### DESCRIPTION OF THE DRAWINGS

The invention will now be described in detail with reference to the Figures in which:

FIG. 1A illustrates a sensor according to an embodiment of the invention in which a layer of fluorinated material is formed on a single layer or multilayer structure;

FIG. 1B illustrates a sensor according to an embodiment of the invention wherein a fluorinated monolayer is formed at the surface of a monolayer or multilayer structure;

FIG. 1C illustrates a sensor according to an embodiment of the invention comprising a bottom-gate TFT supporting a layer of fluorinated material;

FIG. 2 illustrates use of the sensor of FIG. 1C to detect a target analyte; and

FIG. 3 is a graph of light intensity vs wavelength for sensors according to embodiments of the invention in which a fluorinated surface.

### DETAILED DESCRIPTION OF THE INVENTION

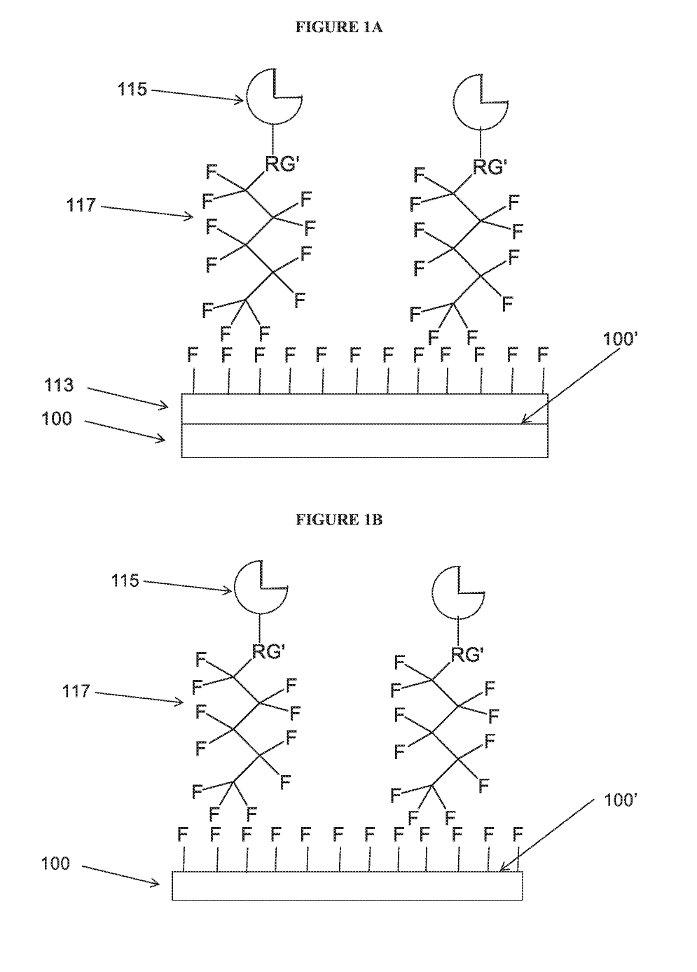

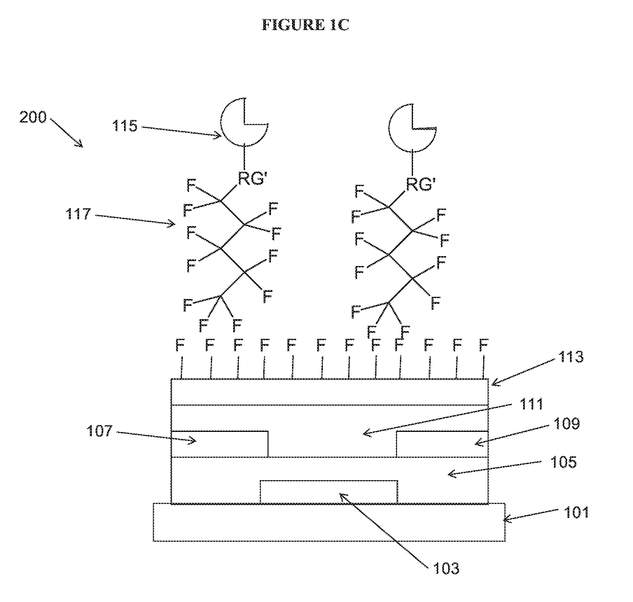

FIG. 1A, which is not drawn to any scale, illustrates a sensor, preferably a biosensor.

The sensor comprises a fluorinated surface provide by fluorinated layer 113 supported on a surface 100′ of a monolayer or multilayer structure 100. The fluorinated layer 113 comprises or consists of a fluorinated material, preferably a fluorinated polymer. The fluorinated layer may have a thickness in the range of 5 nm to 10 microns.

Receptors 115 are bound to fluorinated groups 117 which in turn are bound to fluorinated layer 113. The fluorinated group illustrated in FIG. 1B is a perfluoroalkyl group, however it will be appreciated that other fluorinated groups may be used.

The fluorinated group 117 is bound to the receptor 115 by a group RG′ which is a residue of a reactive group RG that is reacted with the receptor, as described in more detail below. In other embodiments, the fluorinated group is covalently bound directly to the receptor.

Non-covalent F—F interaction between the fluorine atoms of the fluorinated group 117 and the fluorine atoms of the fluorinated layer 113 cause the fluorinated group 117 to bind to the fluorinated layer 113, thereby immobilising the receptors 115 on the fluorinated layer 115. A monolayer of the fluorinated receptor may be formed on the fluorinated material.

In order to form the sensor, in one embodiment the receptor may be deposited onto the fluorinated layer 113 from a formulation comprising the fluorinated receptor dissolved or dispersed in a solvent or solvent mixture. In another embodiment, the sensor may be formed by depositing an fluorinated intermediate compound comprising a fluorinated group onto the fluorinated layer from a formulation comprising the fluorinated receptor dissolved or dispersed in a solvent or solvent mixture, and reacting the fluorinated compound or a derivative thereof with a receptor comprising a reactive group to form the fluorinated receptor.

The fluorinated compound as deposited may comprise a reactive group for reaction with a reactive group of a receptor to form the fluorinated receptor, or the fluorinated compound may be functionalised with a reactive group following deposition to form a derivative of the deposited fluorinated compound wherein the derivative is capable of reacting with a reactive group of a receptor.

Preferably, the solvent or solvent mixture of the formulation comprising the fluorinated receptor or the fluorinated compound comprises or consists of water. One or more co-solvents used with water, if present, may be selected from water-miscible solvents to dissolve the fluorinated compound or fluorinated receptor.

The formulation comprising the fluorinated receptor or the fluorinated compound may be deposited by any method including, without limitation, a coating method such and dip-coating or spin-coating, or a printing method. The whole surface area of the fluorinated layer may be exposed to the formulation such that the fluorinated receptor or the fluorinated compound forms a continuous layer extending across the surface area of the fluorinated layer.

The surface of the fluorinated layer 113 may be treated before deposition of the formulation comprising the fluorinated receptor or the formulation comprising the fluorinated compound. The surface may be treated with a surfactant to enhance wetting of the fluorinated layer 113 by the formulation.

A surfactant as described herein is a material that increases wettability of the fluorinated surface by the formulation comprising the fluorinated receptor or fluorinated compound. An increase in wettability may be determined by a reduction in contact angle of the formulation on the fluorinated surface.

The surfactant may be a non-polymeric or polymeric surfactant. The surfactant is preferably an amphiphilic polymer.

Amphiphilic polymers as described herein may be a synthetic or naturally occurring polymer.

Synthetic polymers include homopolymers and polymers comprising two or more different repeat units. Exemplary synthetic polymers comprise vinylalcohol or vinylpyrollidone repeat units.

A synthetic polymer may be substituted with hydrophobic and hydrophilic substituents, optionally non-polar substituents such as hydrocarbyl groups and polar substituents such as polyether groups. Exemplary hydrocarbyl groups are C₁₋₂₀ alkyl groups; unsubstituted C₅₋₂₀ aryl groups; and C₅₋₂₀ aryl groups substituted with one or more C₁₋₁₂ alkyl groups.

The polymer may be a block copolymer comprising at least one hydrophobic block and at least one hydrophilic block.

The amphiphilic polymer is preferably a surface active protein, optionally bovine serum albumin (BSA), casein or gelatin. Bovine serum albumin is preferred.

The surfactant may be an ionic surfactant, for example sodium dodecyl sulfate; or a non-ionic surfactant, for example a polysorbate surfactant, such as polysorbate-20 (also known as Tween®-20).

The formulation comprising the fluorinated receptor or fluorinated compound may comprise one or more additives to reduce or eliminate signals arising from a non-specific binding. A surface active protein, for example BSA, may both increase wettability of the formulation and limit or prevent non-specific binding.

A residue of the surfactant may form on the surface of the fluorinated layer, however it will be appreciated that the binding of the fluorinated receptor or the fluorinated compound to the fluorinated layer is not prevented by the presence of the surfactant. Without wishing to be bound by any theory, any surfactant on the fluorinated surface may not cover the whole area of the fluorinated surface and/or may be displaced by the fluorinated group of the fluorinated compound or the fluorinated receptor.

The fluorinated group may be irreversibly or reversibly bound to the fluorinated layer 113. Preferably, most (preferably >90 mol %) or all of the fluorinated group remains bound to the fluorinated layer upon washing with distilled water for 1 hour at room temperature.

FIG. 1B illustrates a further embodiment in which surface 100′ is a fluorinated surface of the single layer or multilayer structure. Optionally according to this embodiment, the structure 100 comprises or consists of a glass or plastic layer comprising a fluorinated monolayer, optionally a self-assembled fluorinated monolayer, at surface 100′. The fluorinated groups are bound to the surface 100′ as described with reference to binding of the fluorinated groups to a fluorinated layer 113 in FIG. 1A, and the fluorinated surface 100′ may be treated as described with reference to FIG. 1A.

The sensor of FIG. 1A or 1B may be an optical or electrical sensor, and the single layer or multilayer structure 100 may be selected accordingly.

An optical sensor may be configured to detect, or enable detection of, a change in an optical property upon binding of a target analyte to the receptors.

An electrical sensor may be configured to detect, or enable detection of, a change in an electrical property in a layer thereof.

If the sensor is an optical sensor then the structure 100 preferably comprises or consists of a glass or plastic layer.

In another embodiment, the device may consist of the fluorinated layer 113 having the fluorinated receptor bound thereto.

FIG. 1C, which is not drawn to any scale, illustrates a sensor, preferably a biosensor, according to an embodiment of the invention wherein the structure 100 is a multilayer structure comprising a bottom-gate thin film transistor (TFT).

The bottom-gate TFT comprises a gate electrode 103 over a substrate 101; source and drain electrodes 107, 109; a dielectric layer 105 between the gate electrode and the source and drain electrodes; and a semiconductor layer 111 extending between the source and drain electrodes. The bottom-gate TFT may be a n-type or p-type device.

The bottom-gate TFT may consist of the layers described with reference to FIG. 1C or it may comprise one or more further layers. Exemplary further layers include, without limitation, a charge-transporting layer between the source and drain electrodes and the semiconductor layer, for example as described in WO2016/001095; and more than one layer between the source and drain electrodes and the gate electrode, for example as described in US 2011/127504.

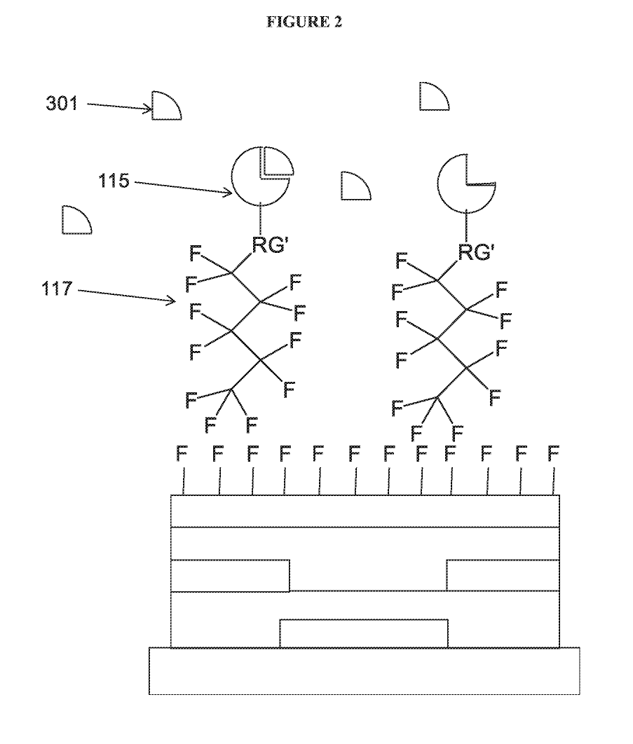

With reference to FIG. 2, in use the sensor 200 is exposed to an analyte fluid, preferably an analyte liquid. If the target analyte 301 is present within the analyte fluid then it may bind to the receptors. Binding of the target analyte to the receptors may cause a change in the threshold voltage (V_{T}) of the device thereby enabling detection of the target.

It will be appreciated that binding of a target analyte may be detected by other electrical changes or optically detectable changes, depending on the structure of the monolayer or multilayer structure 100.

Exemplary analyte fluids include, without limitation: human or animal bodily fluids, optionally a liquid selected from blood, urine, saliva, tears, faeces, gastric fluid, bile, sweat, cerebrospinal fluid and amniotic fluid; cell culture media or other biological samples; food; environmental water, e.g. river, sea or rain water; wine; soil extracts; and gases or other non-biological samples. Exemplary target analytes include, without limitation, DNA, RNA, peptides, carbohydrates, antibodies, antigens, enzymes, proteins, hormones, bacteria, viruses, protozoa, and small molecules, both synthetic and biological.

In the case where the sensor comprises a structure configured to detect a change in an electrical property in a layer thereof, the target analyte is preferably a positively or negatively charged material. Preferably, the target analyte is one or more of bacteria, viruses, protozoa or hormones.

The term ‘small molecules’ as used herein means, in the context of the biological sciences and pharmacology, low molecular weight (typically <900 daltons) compounds that are biologically active. In chemical biology, the term also covers metal ions. Small molecules include, without limitation, lipids, monosaccharides, second messengers and metabolites, as well as drugs and xenobiotics.

### Fluorinated Layer

The fluorinated layer comprises one or more fluorinated materials. Preferably, the fluorinated layer comprises or consists of a fluoropolymer. “Fluoropolymer” as used herein means a polymer comprising at least one fluorinated repeat unit. “Fluorinated repeat unit” as used herein means a repeat unit substituted with one or more substituents selected from fluorine atoms and fluorinated groups. Each substitution position of each carbon atom of a fluorinated repeat unit may be substituted with a substituent selected from a fluorine atom or a fluorinated group, or one or more substitution positions of one or more carbon atoms of a fluorinated repeat unit may be substituted with H or a non-fluorinated substituent.

Exemplary fluorinated groups include, without limitation, partially fluorinated or perfluorinated alkyl groups or aryl groups, optionally phenyl groups.

The fluoropolymer may be a homopolymer of a fluorinated repeat unit or a copolymer, optionally a random, alternating or block copolymer, comprising two or more different repeat units. In the case of a homopolymer, the repeat unit of the polymer is substituted with one or more substituents selected from fluorine atoms and fluorinated groups. In the case of a copolymer, at least one repeat unit of the copolymer is substituted with one or more substituents selected from fluorine atoms and fluorinated groups. A copolymer may comprise two or more different fluorinated repeat units. A copolymer may comprise one or more non-fluorinated repeat units. The fluoropolymer may be a homopolymer consisting of partially fluorinated or perfluorinated repeat units or it may be a copolymer comprising a partially fluorinated or perfluorinated repeat unit and one or more co-repeat units wherein the or each co-repeat unit independently in each occurrence may be non-fluorinated, partially fluorinated or perfluorinated.

Exemplary fluoropolymers include, without limitation, polytetrafluoroethene (PTFE), polyvinylfluoride (PVF), polyvinylidenefluoride (PVDF), polychlorotrifluoroethylene, polyhexafluoropropylene, poly(perfluoropropylvinylether), poly(perfluoromethylvinylether), perfluoroalkoxy polymers, optionally PFA or MFA, polymers of fluorinated ethylene and fluorinated propylene, polymers of ethylene and tetrafluoroethylene, polymers of ethylene and chlorotrifluoroethylene, perfluorinated elastomers, copolymers of chlorotrifluoroethylene and vinylidene fluoride, fluoroelastomers, for example polymers of tetrafluoroethylene and propylene, perfluoropolyethers, perfluoropolyoxetanes and Cytop™.

Fluoropolymers as described herein may be crystalline or amorphous.

Fluoropolymers as described herein suitably have a polystyrene-equivalent weight-average molecular weight (Mw) measured by gel permeation chromatography in the range of about 1×10³ to 1×10⁸, and preferably 1×10⁴ to 1×10⁷.

The fluorinated layer 113 may contain one or more fluoropolymers. Fluoropolymer layer comprises or consists of the one or more fluoropolymers.

The fluorinated layer 113 is preferably formed by depositing a solution comprising the at least one fluorinated material and at least one solvent over the semiconductor layer. The fluorinated layer may be deposited by spin coating.

A fluorinated polymer may be deposited from a solution, or fluorinated monomers may be deposited and then reacted to form a layer of a fluorinated polymer. Fluorinated monomers may be deposited by any suitable method including, without limitation, thermal evaporation and deposition from a solution.

In the case where the sensor comprises a bottom gate TFT, the fluorinated layer 113 is preferably in direct contact with the semiconductor layer. The fluorinated layer 113 may form a barrier to ingress of moisture and/or oxygen into the TFT 100 as well as providing the outer surface for binding of the fluorinated group.

In the case where the fluorinated layer is a polymer, it may be crosslinked. Crosslinking may improve barrier properties of the layer of fluorinated polymer as compared to an uncrosslinked layer.

A fluorinated polymer may be substituted with crosslinkable substituents and/or may be blended with a crosslinking material. The fluorinated polymer may be crosslinked after its deposition from a solution, or crosslinking may occur during polymerisation in the case where fluorinated monomers are deposited and polymerised.

### Fluorinated Receptor

The fluorinated group is bound to the receptor to form a fluorinated receptor, either before or after binding of the fluorinated group to the fluorinated surface. The fluorinated group is preferably covalently bound to the receptor.

Fluorinated group 117 of the fluorinated receptor may by formed by reaction of a fluorinated compound of formula (I) with the receptor:

FG-RG (I)

wherein FG is the fluorinated group and RG is a reactive group capable of reacting with a reactive group of the receptor to form a covalent bond between the fluorinated group and the receptor. It will be understood that the intermediate fluorinated compound of formula (I) does not comprise a biomolecule.

Any bioconjugation method may be used in the case where the molecular recognition element is a biomolecule, optionally click chemistry.

Preferably, the compound of formula (I) is soluble in water or a mixture of water and one or more water miscible solvents. Preferably, RG is an ionic group or a group capable of hydrogen bonding, for example a group comprising at least one N—H, S—H, hydroxyl or COOH group.

RG may comprise or consist of one of the following pairs of groups for forming a covalent bond, the receptor comprising the other of the pair: thiol-maleimide; alkyne-azide; alkene-azide; alkyne-nitrone; alkene-tetrazine; and alkene-tetrazole.

In another embodiment, the fluorinated group may be introduced during synthesis of the receptor, in which case the fluorinated group may be bound directly to the receptor or may be bound through a residue RG′ formed from a reactive group RG.

Optionally, the fluorinated group is a partially fluorinated or perfluorinated C₁₋₂₀ alkyl group.

A wide range of receptors are known to the skilled person. The receptor is preferably a biomolecule. Exemplary biomolecules include, without limitation:

biological material, optionally peptides, carbohydrates, antibodies, antigens, enzymes, proteins, cell receptors, DNA, RNA, PNA, aptamers and natural products;
biologically derived material, optionally recombinant antibodies, engineered proteins; and
biomimics, optionally synthetic receptors, biomimetic catalysts, combinatorial ligands and imprinted polymers.

In order to form the fluorinated receptor, the receptor may be functionalised, if required, with a reactive group capable of reacting with reactive group RG of a compound of formula (I).

### Thin Film Transistor

The semiconductor layer of the thin film transistor may comprise or consist of any semiconducting material including, without limitation, silicon, an organic semiconducting material, graphene or carbon nanotubes. Organic semiconductors include polymers and non-polymeric compounds. The semiconductor layer may comprise a blend of a non-polymeric organic semiconductor and a polymer. Exemplary organic semiconductors are disclosed in WO 2016/001095, the contents of which are incorporated herein by reference. Preferably, the semiconducting material is an organic semiconducting material and the TFT is an organic TFT.

An organic semiconducting layer may be deposited by any suitable technique, including evaporation and deposition from a solution comprising or consisting of one or more organic semiconducting materials and at least one solvent. Exemplary solvents include mono- or poly-alkylbenzenes such as toluene and xylene; tetralin; and chloroform. Solution deposition techniques include coating and printing methods, for example spin coating dip-coating, ink jet printing, roll printing and screen printing.

The length of the channel defined between the source and drain electrodes may be up to 500 microns, but preferably the length is less than 200 microns, more preferably less than 100 microns, most preferably less than 20 microns.

The semiconducting layer extends between the source and drain electrodes and may extend over the source and/or drain electrodes.

The gate electrode can be selected from a wide range of conducting materials for example a metal (e.g. gold) or metal compound (e.g. indium tin oxide). Alternatively, conductive polymers may be deposited as the gate electrode. Such conductive polymers may be deposited from solution using, for example, spin coating or ink jet printing techniques and other solution deposition techniques discussed above.

The insulating layer comprises a dielectric material. Preferably, the dielectric constant, k, of the dielectric is at least 2 or at least 3. The dielectric material may be organic or inorganic. Preferred inorganic materials include Si0₂, SiNx and spin-on-glass (SOG). Preferred organic materials are generally polymers and include insulating polymers such as poly vinylalcohol (PVA), polyvinylpyrrolidine (PVP), acrylates such as polymethylmethacrylate (PMMA) and benzocyclobutanes (BCBs). The insulating layer may be formed from a blend of materials or comprise a multi-layered structure.

The dielectric material may be deposited by thermal evaporation, vacuum processing or lamination techniques as are known in the art. Alternatively, the dielectric material may be deposited from solution using, for example, spin coating or ink jet printing techniques and other solution deposition techniques discussed above. If the dielectric material is deposited from solution then the dielectric material should not be dissolved if an organic semiconductor is deposited onto it from solution. Techniques to avoid such dissolution include: use of orthogonal solvents for example use of a solvent for deposition of the organic semiconducting layer that does not dissolve the dielectric layer; and cross linking of the dielectric layer before deposition of the organic semiconductor layer. The thickness of the insulating layer is preferably less than 2 micrometres, more preferably less than 500 nm.

For ease of manufacture the source and drain electrodes are preferably the same material, preferably a metal which may be deposited by any suitable method, optionally thermal evaporation.

The sensor has been described herein with reference to a sensor in which the single layer or multilayer structure is a bottom gate TFT. It will be appreciated that other single layer or multilayer structures may be used to form a sensor capable of sensing a change, or enabling detection of a change, upon binding of a target analyte to the receptors. The sensor may be a capacitive sensor, for example where the single layer or multilayer comprises an interdigitated electrode array sensor; an electrochemical sensor; or an optical sensor.

### Applications

Applications of the sensor as described herein include, without limitation: pathogen detection, diagnostics, detection of disease related biomarkers, environmental monitoring, food safety control and military purposes. For example,
Glucose monitoring in diabetes patients
Detection of pesticides and river water contaminants
Remote sensing of airborne bacteria e.g. in counter-bioterrorist activities
Determining levels of toxic substances before and after bioremediation
Detection of organophosphate
Detection of electron acceptors, e.g. trinitrotoluene
Measurement of folic acid or vitamin B12
Determination of drug residues in food, such as antibiotics and growth promoters, particularly meat and honey
Drug discovery and evaluation of biological activity of compounds
Detection of toxic metabolites such as mycotoxins

### EXAMPLES

### Example 1—Formation of a Fluorinated Receptor

A 19-base oligonucleotide with fluorescein at the 5′ terminus and a thiol at the 3′ terminus was dissolved in 0.1M dithiotrietol in sodium phosphate buffer (0.1 M, pH 7.4) to a DNA conc. of 75 uM. This solution was left to incubate at RT for 1 h and then desalted by filtering through a NAP-25 column, eluting with sodium phosphate buffer (0.1 M, pH 7.4). 150 uL of a 2 mM solution of N-(heptadecafluoroundecyl)-maleimide in DMSO was added to 1350 uL of the thus filtered DNA solution. The reaction mixture was left to incubate at RT for 1 h, then excess linker was removed using a NAP-25 size exclusion column, eluting with 1:9 DMSO/sodium phosphate buffer (0.1M, pH 7.4). The final concentration of fluorous-tagged DNA was then adjusted to 20 uM by diluting with the same solvent system.

### Example 2—Immobilisation of the Fluorinated Receptor (No BSA Surface Treatment)

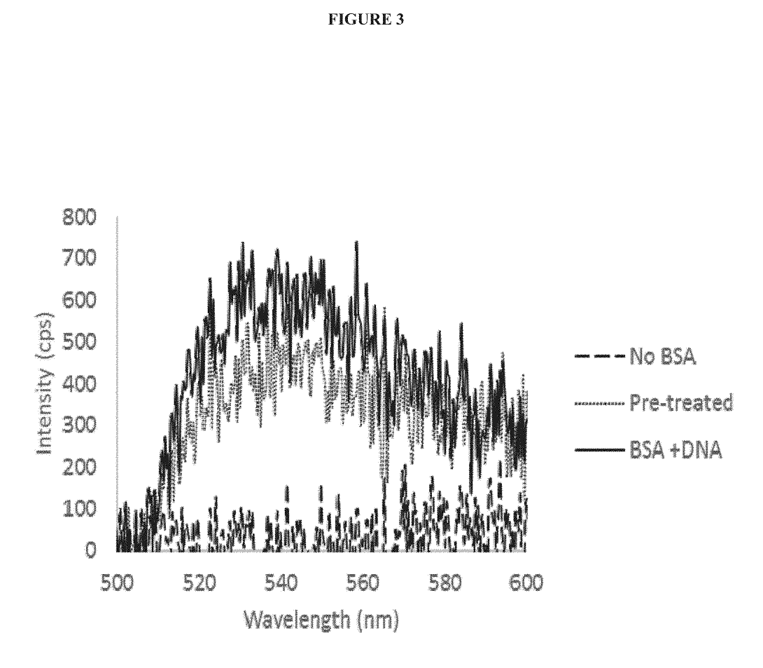

A 1 mL droplet of the fluorous-tagged DNA solution prepared in Example 1 was deposited on the surface of a film of PTFE (approx. 100 nm, formed via spin-coating) and was left to incubate for 1 h at RT. The surface was then washed ×3 with sodium phosphate buffer and ×3 with water, before drying with compressed nitrogen. The surface was interrogated for the presence of fluorescein-tagged DNA using an optical microscope equipped with a spectrometer (exciting at 480 nm). No fluorescence was observed using this method (FIG. 3), indicating that DNA immobilisation was unsuccessful.

### Example 3—Immobilisation of the Fluorinated Receptor (Surface Pre-Treated with BSA)

A 75 uL droplet of 3% bovine serum albumin (BSA) in sodium phosphate buffer was deposited on the surface of a PTFE film (formed as described in Example 2) and incubated at RT for 10 min (treated region controlled using 9 mm diameter, 1 mm depth silicon isolator, Grace Biolabs). The treated region was washed ×3 with the same buffer, then 75 uL of the fluorous-tagged DNA solution prepared in Example 1 was deposited on the treated area and was incubated at RT for 1 h. The treated area was then washed ×3 with sodium phosphate buffer and ×3 with water, followed by drying with compressed N2. The surface was integrated using an optical microscope (FIG. 3), as described in Example 2, which indicated that the fluorous-tagged DNA had been uniformly distributed on the BSA-treated region of the fluoropolymer film.

### Example 4—Immobilisation of the Fluorinated Receptor (Direct Treatment with DNA+BSA)

3% w/v BSA was dissolved in the fluorous-tagged DNA solution prepared in Example 1 and a 75 uL droplet of the resultant solution was deposited on the surface of a PTFE film (formed as described in Example 2; treated region controlled using 9 mm diameter, 1 mm depth silicon isolator, Grace Biolabs). After incubation at RT for 1 h, the treated region was washed ×3 with sodium phosphate buffer and ×3 with water, then dried with compressed nitrogen. The surface was integrated using an optical microscope (FIG. 3), as described in Example 2, which indicated that the fluorous-tagged DNA had been uniformly distributed on the BSA-treated region of the fluoropolymer film.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A method of forming a sensor comprising a single layer or multilayer structure; a fluorinated layer having a fluorinated surface on the single layer or multiple layer structure; and a receptor having a fluorinated group on the fluorinated surface, the method comprising treating the fluorinated surface with a surfactant and either:
depositing the receptor having a fluorinated group onto the fluorinated surface from a formulation comprising one or more solvents in which the receptor is dissolved or dispersed; or
depositing a fluorinated compound comprising a fluorinated group onto the fluorinated surface from a formulation comprising one or more solvents in which the fluorinated compound is dissolved or dispersed, and reacting the fluorinated compound or a derivative thereof with a receptor comprising a reactive group to form the receptor having the fluorinated group.

2. A method according to claim 1 wherein the formulation comprises the surfactant.

3. A method according to claim 1 wherein the fluorinated surface is treated with the surfactant before deposition of the fluorinated compound or fluorinated receptor.

4. A method according to claim 1 wherein the surfactant is an amphiphilic polymer.

5. A method according to claim 1 wherein the surfactant is a surface active protein.

6. A method according to claim 5 wherein the surface active protein is bovine serum albumin.

7. A method according to claim 1 wherein the one or more solvents comprise or consist of water.

8. A method according to claim 1 wherein the fluorinated layer is a layer of fluorinated material deposited on a surface of the single layer or multilayer structure.

9. A method according to claim 8 wherein the fluorinated layer has a thickness in the range of 5 nm-10 microns.

10. A method according to claim 8 wherein the fluorinated layer comprises a fluorinated polymer.

11. A method according to claim 8 wherein the fluorinated polymer is crosslinked.

12. A method according to claim 1 wherein the fluorinated surface is a fluorinated self-assembled monolayer formed at a surface of the single layer or multilayer structure.

13. A method according to claim 1 wherein the fluorinated group is a perfluoroalkyl group.

14. A method according to claim 1 wherein the receptor is selected from DNA, PNA, RNA, peptides, carbohydrates, antibodies, antigens, enzymes and proteins.

15. A sensor comprising:
a structure configured to detect a change in an electrical property in a layer thereof;
a fluorinated layer on a surface of the structure; and
a receptor bound to a fluorinated group on an outer surface of the fluorinated layer.

16. A sensor according to claim 15 wherein the detector is a thin film transistor comprising a gate electrode; source and drain electrodes; a dielectric layer between the gate electrode and the source and drain electrodes; and a semiconductor layer extending between the source and drain electrodes.

17. A sensor according to claim 16 wherein the thin film transistor is a bottom gate thin film transistor.

18. A sensor according to claim 16 wherein the thin film transistor is an organic thin film transistor.

19. A sensor according to claim 16 wherein the fluorinated layer is adjacent to and in contact with the semiconductor layer.

20. A sensor comprising:
a structure configured to detect a change in an optical property in a layer thereof;
a fluorinated layer on a surface of the structure; and
a receptor bound to a fluorinated group on an outer surface of the fluorinated layer.
